(19) United States
(12) Patent Application Publication
(10) Pub. No.: US 2008/0206758 A1
(43) Pub. Date: Aug. 28, 2008

(54) POLYNUCLEIC ACID-ATTACHED PARTICLES AND THEIR USE IN GENOMIC ANALYSIS

(75) Inventor: Gary W. Loge, State College, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: LCM Technologies, Inc., State College, PA (US)

(21) Appl. No.: 11/872,892

(22) Filed: Oct. 16, 2007

Related U.S. Application Data
(60) Provisional application No. 60/829,719, filed on Oct. 17, 2006.

(51) Int. Cl. C12Q 1/68; C07H 21/00

(52) U.S. Cl. 435/6; 536/25.3; 536/25.32

(57) ABSTRACT

Disclosed are methods for preparing particle-linked polynucleotides, and using the particle linked polynucleotides in genomic analysis. The particles as disclosed are characterized as having a size variance of less than 2%.

### CROSS-REFERENCE TO RELATED APPLICATIONS

This application claims the benefit of U.S. Provisional Application No. 60/829,719 filed Oct. 17, 2006, the entirety of which is incorporated by reference herein.

### FIELD OF THE INVENTION

The field of the invention includes polynucleic acids, nanoscale and microscale particles, and genomic analytical techniques. The field of the invention also includes methods of size fractionating nanoscale and microscale particles.

### BACKGROUND OF THE INVENTION

Deoxyribonucleic acids (DNA) are analyzed for a variety of applications, such as identifying unknown parasites, identifying genetic-based diseases in humans and animals, and conducting research. For example, the forensic community uses the short tandem repeat (“STR”) method for identifying individuals based on DNA evidence. The STR method analyzes tetra- and penta-nucleotide repeats, and is an improvement over earlier methods for a number of reasons: i) STR provides increased sensitivity (0.5 to 2 ng DNA sample required), ii) STR is better able to analyze degraded DNA (but not ideal as discussed below), and iii) STR is able to maintain the statistical robustness required for determining identity (approximately 1 in 10¹³ to about 1 in 10¹⁸, depending on the number of loci applied). Unfortunately, STR requires as long as a single day to perform complete analysis, and STR has limited use with degraded samples.

Mini-STR is an improvement over standard STR for analyzing degraded DNA samples. Mini-STR fragments are generated using primers that hybridize on the genomic DNA closer to the STR repeat region. Mini-STR generally uses significantly shorter fragments, yet provides similar identification information, as does STR. As a result, mini-STR provides an increased ability to obtain profiles from degraded DNA obtained from skeletal samples. Further improvements in STR analysis are needed.

The U.S. Federal Bureau of Investigation (“FBI”) has included thirteen core STR loci in their Combined DNA Indexing System (“CODIS”) for convicted offenders. As a result of this, several commercial multiplexed STR kits with dye-labeled primers and data analysis software have become available. As state crime laboratories validated these new STR procedures and ramped up their STR analysis capabilities, they found that they were facing an ever-increasing backlog of convicted offender samples and forensic cases. States continue to look for ways to further automate their processes, and improve throughput. Even with the technological improvements in STR analysis, state legislatures continue to add criminal offenses that qualify for CODIS. This has resulted in a continuing backlog of convicted offender samples and non-suspect cases. Accordingly, there is a continuing need to improve efficiency while maintaining rigorous quality standards in STR analysis.

### SUMMARY OF THE INVENTION

A technology that offers improvements to STR analysis is described herein. End-labeled free-solution electrophoresis (“ELFSE”) uses a high mass particle linked to a primer or related polynucleic acid that functions “drag-label”, which is added to a polymerase chain reaction (“PCR”) amplification product to provide a near constant drag coefficient independent of DNA fragment size. This allows separation by native charge on the DNA fragments in a simple electrolytic buffer, resulting in significantly shorter separation times than capillary gel electrophoresis. ELFSE STR analysis can be run on standard capillary instrumentation, using dye sets similarly used in commercially available STR kits. In addition, the drag-labels described herein have a dramatically increased fluorescence sensitivity which can be advantageously used to uniquely label substantially all STR loci. This allows unambiguous identity of loci even when the range of allele sizes overlap. These improvements enable improved mini-STR multiplexing, which can be useful in obtaining DNA profiles from degraded samples.

Accordingly, one aspect of the present invention provides methods of preparing neutralized polynucleic acid-attached particles, comprising: size fractionating a plurality of charged surface-functionalized particles to provide a plurality of size-fractionated charged surface-functionalized particles being characterized as having a size variance of less than 2 percent; neutralizing at least a portion of the size-fractionated charged surface-functionalized particles with a hydrophilic neutralizing agent to give rise to neutralized size fractionated particles being characterized as having a hydrophilic surface; and attaching a polynucleic acid, directly or indirectly, to each of at least a portion of the neutralized size fractionated particles, or to each of at least a portion of the size-fractionated charged surface-functionalized particles, to provide neutralized polynucleic acid-attached particles.

In other aspects, the present invention provides methods of analyzing particle linked amplicons comprising: providing a plurality of primer-linked particles comprising PCR primer molecules, each labeled with a neutralized particle characterized as having an average particle size in the range of from about 5 nm to about 100 nm, each particle having a size variance of less than about 0.5 percent; and amplifying a DNA sample using the primer-linked particles in the presence of reagents to give rise to particle-labeled amplicons; and analyzing the particle-linked amplicons.

Additionally there are provided polynucleic acid analysis kits, comprising a plurality of surface neutralized particles having an average particle size in the range of from about 5 nm to about 500 microns, the size variance of the particles being less than about 0.5 percent, at least a portion of the particles being attached to one linker molecule; and a reagent to link a polynucleic acid to each of the linker molecules.

In other aspects, the present invention provides particles, comprising a plurality of surface neutralized particles having an average particle size in the range of from about 5 nm to about 500 microns, the size variance of the particles being less than about 0.5 percent, wherein at least a portion of said particles each capable of being attached to a polynucleic acid.

Also, the present invention provides methods of analyzing DNA fragments by size, comprising: attaching a plurality of surface neutralized particles to sample DNA fragments, wherein the surface neutralized particles having an average particle size in the range of from about 5 nm to about 500 microns, the size variance of the particles being less than about 0.5 percent, wherein at least a portion of said particles each capable of being attached to a polynucleic acid to provided DNA-linked particles; and analyzing the DNA-linked particles using ELFSE.

The drag-label characteristics used in ELFSE STR analysis include low size-variance to give homogeneous drag and low native charge to avoid interfering with native charge on the DNA fragments. These characteristics have been demonstrated for the drag-labels made according to certain embodiments of the present invention. Improvements in reduced size-variance of the drag label particles for STR analysis are needed. In addition to these improvements, methods to attach to STR primers, allelic ladder and internal lane size standards are described. After development of suitable drag-labeled STR primers and demonstrated ability to call alleles are completed, blind concordance studies and elements of developmental validation studies are conducted using commercially-available capillary instrument. In addition, fragment size software can be provided to handle ELFSE data, which has migration times inversely proportional to DNA native charge.

The general description and the following detailed description are exemplary and explanatory only and are not restrictive of the invention, as defined in the appended claims. Other aspects of the present invention will be apparent to those skilled in the art in view of the detailed description of the invention as provided herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 is a flow chart depicting several embodiments of the present invention;

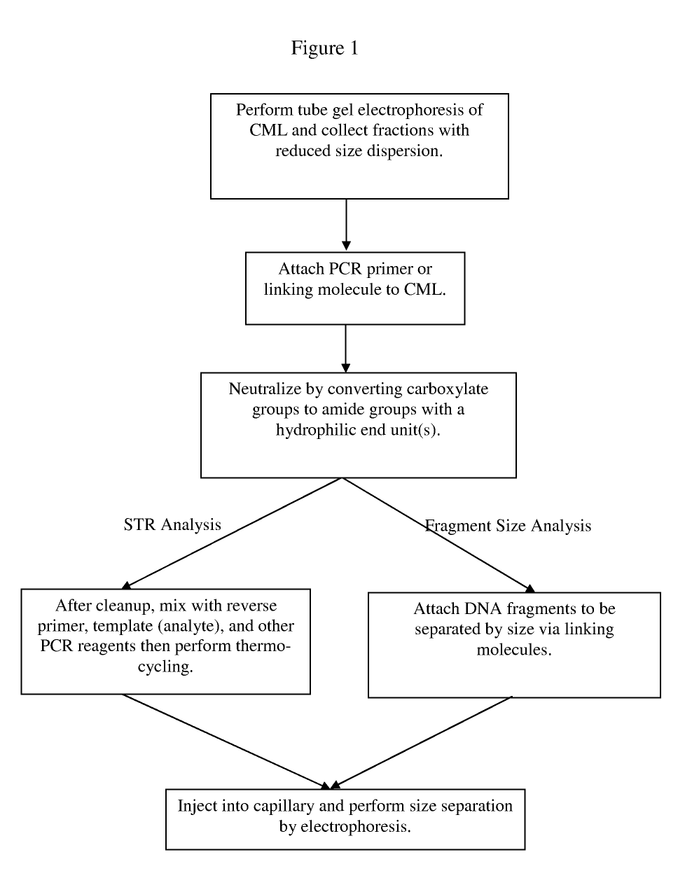

FIG. 2 illustrates a surface of an embodiment of a carboxylate-modified surface latex sphere with high density surface charge;

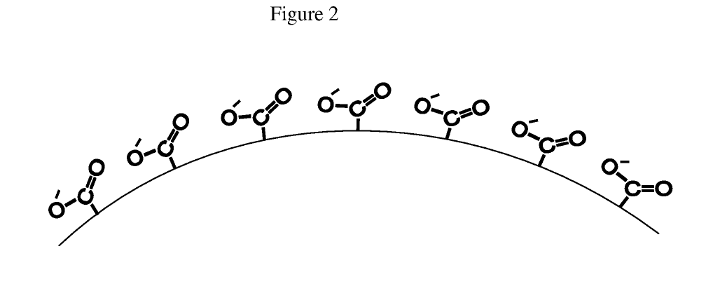

FIG. 3 illustrates a hydrophilic amide-modified surface latex sphere prepared using the carboxylate-modified surface shown in the previous diagram and 3-aminopropanol;

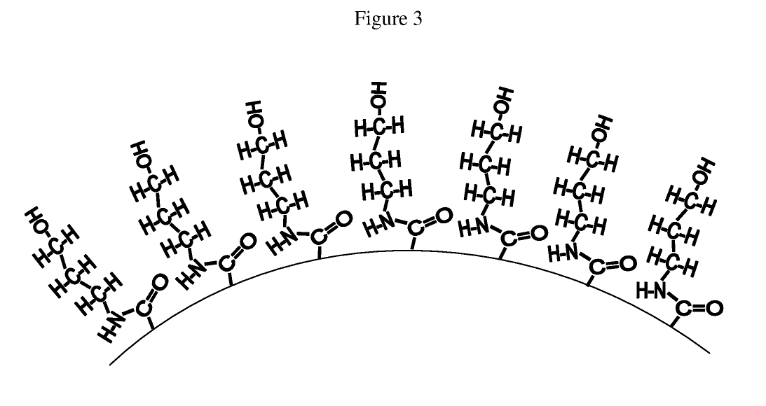

FIG. 4 illustrates an embodiment of a suitable to be gel electrophoresis system used in the present invention;

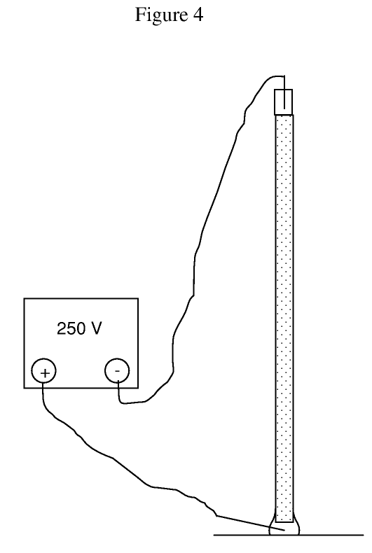

FIG. 5 depicts Capillary Electrophoresis of 43 nm latex sample (0.001% w/w in water) with no fractionation using a 2 minute-10 cm hydrodynamic injection into a coated capillary containing 1×TBE buffer;

FIG. 6 illustrates capillary electrophoresis of 43 nm latex sample after fractionation performed in 0.7% agarose gel using fractional collection;

FIG. 7 illustrates fluorescence intensity vs. migration time for capillary electrophoresis of size fractions collected with fraction numbers noted; the upper three traces were taken a day later than the lower five traces; and

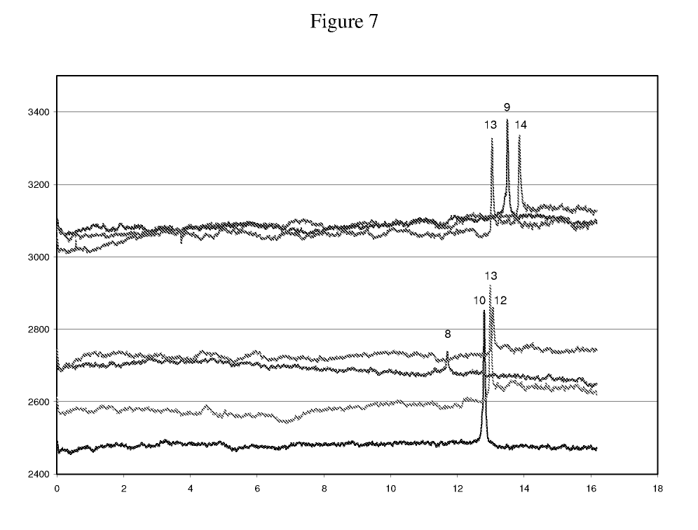

FIG. 8 illustrates chemical linking of PCR primer onto a CML particle surface. A similar linking mechanism can be used to attach a linker molecule for later addition of the DNA fragment to be characterized for size.

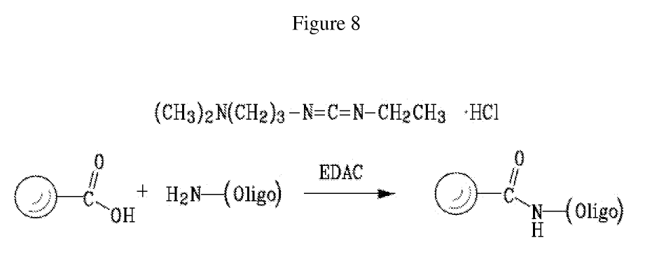

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

The development and use of STR loci is described in U.S. Pat. No. 5,364,759 and commercialized by the Promega Corporation. U.S. Pat. No. 5,599,666 describes allelic ladders for the loci CSF1PO, F13A01, FESFPS, LPL and vWA. Use of the GenePrint™ STR System uses a polymerase chain reaction (PCR), which is described in U.S. Pat. Nos. 4,683,195, 4,965,188 and 4,683,202. Each of these patents is incorporated by reference herein in their entirety.

Use of Free-Solution Electrophoresis with Particle Drag-Labels. Capillary gel electrophoresis is used for high-throughput genomic analysis including sequencing, SNP discovery, mutation detection, and fragment size analysis of STR loci. Sieving matrices used in capillary gel electrophoresis often have separation efficiency limits. To obtain high resolution with a sieving matrix, a minimum separation distance of about 36 cm can be used. It also typically uses longer run times, since analytes must traverse the capillary length through a viscous sieving matrix. The sieving matrix separation efficiency tends to decrease when the electric field strength is higher than about 200 V/cm, which imposes a minimum run time for maximum separation efficiency when minimum capillary length are desired.

One aspect of the invention provides an alternative type of capillary electrophoresis not limited by sieving matrix. This aspect uses a particle that functions as a “drag-label” in free-solution to give essentially equal drag to all polynucleic acid fragments, allowing separation by polynucleic acid charge. Without being bound by any theory of operation, migration is through a relatively low viscosity buffer, so free-solution electrophoresis can be used to decrease run-time by a factor of three compared to capillary gel electrophoresis (e.g., 10 min. for free-solution electrophoresis versus 30 min. for gel electrophoresis). In addition, free-solution electrophoresis does not appear to be limited by electric field strength, so higher field strengths can be used to further increase the migration rate and decrease run time. Field strengths and the rate at which the detector can acquire data can be controlled to decrease the run-time for free-solution electrophoresis.

In addition to improvements in run time arising from reduced viscosity and higher field-strengths, shorter separation distances can be accommodated with no loss in separation resolution, while gel electrophoresis typically uses a minimum capillary length of about 36 cm for optimum separation efficiency. Shorter separation distances can decrease the run time proportionally to the inverse square of the length, using a similar voltage (e.g., higher electric fields can be used for shorter lengths).

A micro-fluidic chip can separate short DNA fragments (12 bases) in free solution as part of a “lab-on-a-chip” application. Free-solution electrophoresis on a micro-fluidic chip can be combined with DNA isolation from samples and PCR amplification. Chip technology has the additional benefit of requiring only nanoliter reagent volumes. ELFSE can be used to read-lengths up to 130 bases using streptavidin as the drag-label. ELFSE can be used for genotyping using short fragments (10-20 bases). ELFSE can replace capillary gel electrophoresis for genetic fragment size analysis by using suitable “drag-label” such as the particles described herein. A suitable drag-label is characterized as having a uniform size i.e., minimal size homogeneity dispersion. A suitable drag-label may also be characterized as having a drag coefficient comparable to that of the largest DNA fragment to be separated.

A variety of particles, such as latex nanospheres, can be used as a suitable drag-label to perform ELFSE for STR analysis and other genomic analysis applications. A suitable procedure for obtaining useful drag-labels includes size-fractionating carboxylate-modified latex (CML) nanospheres. CML nanospheres are a common form of latex particle characterized as having colloidal stability and can be readily conjugated with bio-molecules such as poly-nucleic acids. Size-fractionated latex nanospheres can be further processed to eliminate or minimize surface charges that could otherwise interfere with ELFSE migration rates.

One aspect of the present invention provides methods of preparing neutralized polynucleic acid-attached particles. These particles, as described herein, can be used in a variety of genomic analytical techniques, such as DNA forensics. These methods include size-fractionating a plurality of charged surface-functionalized particles to provide a plurality of size-fractionated charged surface-functionalized particles being characterized as having a size variance of less than 2 percent. At least a portion of the size-fractionated charged surface-functionalized particles are neutralized with a hydrophilic neutralizing agent to give rise to neutralized size fractionated particles being characterized as having a hydrophilic surface. A polynucleic acid can be attached, directly or indirectly, to each of at least a portion of the neutralized size-fractionated particles, or to each of at least a portion of the size-fractionated charged surface-functionalized particles subsequent to neutralization, to provide neutralized polynucleic acid-attached particles.

Particles can be suitably size fractionated using any of a variety of particle classification techniques. Suitable classification techniques in general are capable of separating particles based on differences in electrical charge, particle shape, composition, density, mass, diffusion coefficient, rheology, chemical or biological affinity, as well as any combination thereof. Suitable classification techniques include, for example, electrophoresis, chromatography, field-flow fractionation, as well as combinations of these techniques. Preferred techniques include wherein the plurality of charged surface-functionalized particles are size-fractionated using gel electrophoresis, field-flow fractionation, or a combination of both. Gel electrophoresis for size fractionating the particles can suitably use an agarose gel, acrylamine or acrylamine derivative gel, cellulosic gel, or any combination thereof. The gels are typically low viscosity, low solid weight fraction, which aids in efficient separation time. Nano-fabricated physical sieving devices can also be used, as well as any combinations of the above listed methods. In these embodiments, a plurality of particles dispersed in a medium, such as an aqueous or non-aqueous solvent, can be pumped into a tube containing agarose gel or a suitable nanofabricated physical sieving device, the particles are separated within the tube, and size fractions are collected for subsequent processing.

Charged surface-functionalized latex particles can also contain fluorophores. Suitable fluorophores include fluorescent dye molecules, energy-transfer donor-acceptor fluorescent dye molecule pairs, quantum dots, or any combination thereof. Fluorophores can be particularly useful for identifying polynucleic acids. Fluorescent dye molecules, energy transfer dye molecules, or quantum dots, or any combination thereof, can typically emit at one or more different wavelengths with different intensities resulting in fluorescent pattern signatures. In certain instances, the fluorescent pattern signatures can be used as a bar-code in identifying the polynucleic acid.

The charged surface-functionalized particles suitably comprise charged surface-functionalized latex particles, polymer particles, as well as other surface-functionalized colloidal particles. Suitable particles may be polymeric or non-polymeric. Suitable latex particles include a polymeric material composed of repeating units derived from styrenic monomer, acrylic monomer, acrylonitrile monomer, diene monomer, or any combination thereof. Examples of suitable polymeric particles include polyesters (such as poly(lactic acid), poly(glycolic acid), or poly(lactic-co-glycolic acid)), poly(lactic acid-co-lysine), poly(lactic acid-graft-lysine), polyanhydrides (such as poly(fatty acid dimer), poly(fumaric acid), poly(sebacic acid), poly(carboxyphenoxy propane), poly(carboxyphenoxy hexane), copolymers of these monomers and the like), poly(anhydride-co-imides), poly(amides), poly(ortho esters), poly(iminocarbonates), poly(urethanes), poly(organophasphazenes), poly(phosphates), poly(ethylene vinyl acetate) and other acyl substituted cellulose acetates and derivatives thereof, poly(caprolactone), poly(carbonates), poly(amino acids), poly(acrylates), polyacetals, poly(cyanoacrylates), poly(styrenes), poly(vinyl chloride), poly(vinyl fluoride), poly(vinyl imidazole), chlorosulfonated polyolefins, polyethylene oxide and copolymers and blends thereof, as well as any combination of these polymers or corresponding monomers giving rise to one or more copolymers. In addition, suitable charged surface-functionalized particles can also include one or more functional groups at the particle surface. The functional groups are useful for attaching a polynucleic acid. Suitable surface functional groups include carboxylic acids, aliphatic amines, aromatic amines, hydrazides, sulfates, as well as any combination thereof. Functional groups can be provided at the particle surface using polymerization, surface treatment, as well as grafting of acidic or basic functional groups to the particles.

The plurality of charged surface-functionalized particles prior to fractionating is characterized as having an average particle size in the range of from about 5 nm to about 500,000 nm. Average particle sizes can even be greater than about 10 nm, or greater than about 20 nm, or even greater than about 50 nm. Average particle sizes can be smaller than 300,000 nm, or even smaller than about 100,000 nm, or even smaller than about 50,000 nm, or even smaller than about 30,000 nm, or even smaller than about 10,000 nm, or even smaller than about 5,000 nm, or even smaller than about 3,000 nm, or even smaller than about 1,000 nm, or even smaller than about 500 nm, or even smaller than about 300 nm, or even smaller than about 200 nm, or even smaller than about 150 nm, or even smaller than about 100 nm, or even smaller than about 80 nm, or even smaller than about 60 nm, or even smaller than about 40 nm, or even smaller than about 30 nm. These average particle sizes also describe the average particle sizes of the size fractionated particles, as well as the particles having attached polynucleic acids.

The charged surface-functionalized particles prior to size fractionating are suitably characterized as having a size variance of greater than about 2 percent, sometimes, even as high as 5 percent, or even as high as 15 percent, or higher. For a given particle size distribution, the size variance is defined as the standard deviation of the particle size distribution divided by the average size of the particle size distribution. When expressed as a percentage, this value of the size variance is multiplied by 100%. In some embodiments, the plurality of charged surface-functionalized particles prior to fractionating are suitably characterized as having an average particle size in the range of from about 5 nm to about 500,000 nm and a size variance distribution ranging from about 2% to about 20% of the average size.

Particle size can be measured using one or more of a variety of methods such as microscopy and scattering techniques. Suitable methods of determining particle size include transmission electron microscopy, scanning electron microscopy, atomic force microscopy, light scattering, optical microscopy, x-ray scattering, neutron scattering, dynamic light scattering, or any combination thereof. Size variance of polynucleic-acid-linked particles can be measured according to dynamic light scattering, ELFSE separation of DNA size ladder, as well as combinations thereof.

Size fractionation reduces the size variance of the particles. For example, the plurality of size-fractionated charged surface-functionalized particles are suitably characterized as having a size variance of less than about 2 percent, or less than about 1 percent, or less than about 0.5 percent, or less than about 0.1 percent, or even less than about 0.05 percent.

Neutralization. The charged size-fractionated surface-functionalized particles can be neutralized by converting at least a portion of charged surface functional groups to an uncharged group. The uncharged group includes one or more hydrophilic groups. Suitable hydrophilic groups include a hydroxyl group, amide group, aldehyde group, thiol group, or any combination thereof. The charged size-fractionated surface-functionalized particles can be neutralized by reacting at least a portion of the charged surface functional groups with a hydroxyl-bearing compound, amide-bearing compound, aldehyde-bearing compound, thiol-bearing compound, or any combination thereof. In a particular embodiment, the hydroxyl-bearing compound comprises 3-aminopropanol. Although complete neutralization is preferred, a small amount of charged particles may be acceptable. A suitable degree of neutralization of the particles can be ascertained using electrophoresis. For example, effectively neutralized particles will have essentially no mobility compared to charged particles when placed in an electric field. Accordingly, an electrophoresis band of neutralized size-fractionated surface-functionalized particles will have an intensity less than about 10 percent, or even less than 5 percent, or even less than 1% or less than the intensity of the corresponding electrophoresis band of the charged size-fractionated particles before neutralization. This reduction in charge of the particles can help reduce the electric-field induced mobility of the particles. Accordingly, it is desired that the neutralized beads will not migrate in electrophoresis without DNA attached.

A suitable polynucleic acid can be attached directly to at least a portion of the size-fractionated surface-functionalized particles via covalent bonding before neutralization. Alternatively, each polynucleic acid can be attached indirectly to at least a portion of the neutralized size-fractionated particles via a linker molecule attached to the size-fractionated charged surface-functionalized particles between before neutralization. To ensure that no more than one polynucleic acid is attached to the neutralized size-fractionated surface-functionalized particles, a five-fold excess of size-fractionated surface-functionalized particles can be used so that at least 80% will contain no polynucleic acid or linker molecule. Suitable linker molecules include difunctionalized primary amines for bonding to carboxy or other acidic surface modification, or primary amines comprising carboxylic acid for amine surface-modification or any combination thereof. Suitable difunctionalized primary amines include any compound having the structure

X′—Y—X″

wherein X′ and X″ are functional groups and Y is an alkyl group comprising from 1 to about 40 carbon atoms and from 2 to about 80 hydrogen atoms. X′ and X″ are suitably selected from carboxylate, amine, sulfate, or thiol groups, or any combination thereof. Y is suitably selected to be an alkyl group comprising from 3 to about 10 carbon atoms and from 6 to about 20 hydrogen atoms. The linker molecules can be readily attached to the size-fractionated surface-functionalized particles. For example, a diimide can be used, such as N-ethyl-N′-(3-dimethylamino-propyl)carbodiimide hydrochloride (“EDAC”), to form unstable acyl intermediates that can react rapidly with a primary amine to form an amide. In one method, the concentration of such a primary amine linker molecule is desirably used in much lower concentration than that of the particle concentration to obtain no more than one linker molecule per particle.

In other embodiments, at least a portion of the charged size-fractionated surface-functionalized particles can be neutralized by converting carboxylate groups to amide groups comprising one or more hydrophilic end units. Here, suitable hydrophilic end units include hydroxyls, thiols, amides, or any combination thereof.

Various methods can be used for attaching primers to linker molecules before attachment to the particles. For example, primers can be attached to linker molecules using any of a variety of known chemical attachment methodologies known to those skilled in the art of oligo synthesis. Attaching sample DNA for fragment analysis can also be via a phosphoramidate bond. Further details can be found in Strategies for Attaching Oligonucleotides to Solid Supports, by Devor, E. J., et al., published by Integrated DNA Technologies, Inc., 2005.

Polynucleic acids can be covalently attached to particles with any of several methods. Carboxyl and amino groups are the most common reactive groups for attaching ligands to surfaces. These groups are very stable over time, and their chemistries have been widely explored. Several reactive groups that can be incorporated on the particle surface for covalent coupling include: carboxylic acids, aliphatic amines, aromatic amines, amides, hydrazides, hydroxyls, thiols, and epoxies. For example, attaching an amino group to the 5′ or 3′ end of an oligonucleotide or a PCR primer is straightforward. Amine-modified oligos can then be reacted with carboxylate-modified micro-spheres with carbodiimide chemistry in a one-step process at pH 6-8 (FIG. XX). One typical water-soluble carbodiimide is 1-ethyl-3-(3-dimethylaminopropyl) carbodiimide hydrochloride (“EDAC”), which provides one-step coupling. Another option is to bind biotin-labeled oligos to avidin-coated beads.

At least about 1 percent of the size-fractionated neutral surface-functionalized particles are attached to one polynucleic acid. It is desirable that no more than 10% of the particles with a DNA attached have more than one linker or primer attached. One method to achieve this is by using a five-fold excess of linker or primer relative to the particle number density based on Poisson distribution statistics. This excess can be greater than five-times. Such an excess can give rise to about 80% of the particles having no linker or primer attached, about 18% having one and only one linker or primer attached and about 2% having more than one linker or primer attached. In some embodiments, the solution chemistry can be altered so that at least about 15 percent of the neutralized size-fractionated surface-functionalized particles are attached to one polynucleic acid. In other embodiments, the chemistry can be altered so that more than 20% of the size-fractionated surface-functionalized particles have one polynucleic acid attached.

The methods of preparing the polynucleic acid-linked particles may further include one or more cleaning steps. The cleaning steps can be performed at any point in the method. For example, large particulate materials can be removed using filtration, dissolved gases and light dissolved components may be removed by application of vacuum, and the like. Microfiltration, ultrafiltration and reverse osmosis can be used. The cleaning steps may also include one or more rinsing, washing or dialysis steps. Dialysis is particularly desirable for removing or exchanging dissolved salts and other small molecules, including neutralizing agents. Combinations of any of these cleaning steps can also be carried out.

Polynucleic acid-attached particles useful for STR analysis or mini-STR analysis include a suitable STR primer or a mini-STR primer attached to the particle. A suitable polynucleic acid includes a primer, DNA from a test sample, or RNA from a test sample. In certain applications the particles can be attached directly to DNA fragments obtained from test samples, for example, RFLP fragments of a species to be identified. DNA fragments can be readily attached to a surface-neutralized particle drag label by attaching a linker molecule to a particle before neutralization, and then attaching a DNA fragment to the linker. Suitable primers include any of the know primers known in the art. Suitable primers include a forward primer or a reverse primer. Suitable forward primers can be used in conjunction with a reverse primer for polymerase chain reaction (“PCR”) amplification.

Methods are also provided for making a plurality of primer-linked particles comprising PCR primer molecules wherein the particles have an exceedingly low size variance. In these methods, each of the primers can be labeled with a neutralized particle characterized as having an average particle size in the range of from about 5 nm to about 100 nm, each particle having a size variance less than about 0.5 percent.

PCR amplifying a DNA sample using the primer-linked particles can give rise to particle-labeled amplicons. The PCR amplification process is well known to those in the art of DNA analysis. PCR amplification can include combining reverse primer, a specimen of the polynucleic acid to be analyzed, PCR enzyme, deoxyribonucleotide triphosphates (“dNTPs), buffer containing Mg, with the plurality of PCR primer molecules, each labeled with a neutralized particle to form a polynucleic acid amplification mixture. The mixture can be thermocycled to yield particle-linked amplicons, which are subsequently analyzed. The procedures described herein use particles having very small size variances. For example, the plurality of neutralized polynucleic acid-attached particles has a size variance less than about 0.2 percent, or even less than about 0.1 percent, or even less than about 0.05 percent, or even less than about 0.03 percent.

In PCR a forward-reverse primer pair is specified. About 15 primer pairs specified for STR analysis, which are provided in U.S. Pat. No. 5,364,759, the entirety of which pertaining to STR analysis is incorporated by reference herein. Mini-STR primer pairs exist for all the STR loci as first described in J. Forensic Sci, Vol. 48, No. 5 (2003), pgs. 1054-1064, which is incorporated by reference herein. Additional mini-STR loci have been described in J. Forensic Sci., Vol. 50 (2005)43-53 for the analysis of degraded samples and in Forensic Sci. Int., Vol. 156 (2006), pgs. 242-244 for other international STR loci, which is incorporated by reference herein.

Thermocycling temperatures and conditions using the particle-labeled polynucleic acids are essentially the same as traditional thermocycling without particle-labeled polynucleotides. The number of cycles can vary depending on the sample available and amount of amplification needed. In some embodiments wherein the particles include many dye molecules, the amplicons are readily detected at concentrations much lower than in traditional PCR. Accordingly, fewer thermocycles are needed in certain embodiments involving dyed particles.

The particle-labeled amplicons are readily analyzed using ELFSE. Suitable conditions for conducting ELFSE include temperature between about 5° C. and 40° C., capillary length between about 4 cm and about 100 cm, voltage field strength between about 50 V/cm and about 2000 V/cm, capillary diameter from about 1 micron to about 1000 microns, or even higher, buffer, concentration, and conductivity. The lower range of capillary lengths (e.g., about 4 cm) is readily provided using a microfluidic chip, whereas the higher range can be provided using capillary tubes (e.g., having lengths greater than about 5 cm, or even greater than about 10 cm, or even greater than about 20 cm, or even on the order of about 100 cm). Without being bound by any particular theory of operation, a wide range of voltage field strengths is possible as there appears to be no upper voltage field strength limit except for joule heating and corona arcing.

In addition to ELFSE analysis of particle-labeled amplicons for STR analysis and mini-STR analysis, article-labeled amplicons can be used for RFLP analysis, VNTR analysis, AFLP analysis, kerotyping analysis, mapping analysis of unamplified DNA or RNA samples, or any combination thereof. These methods can provide an improvement over the standard method of fragment analysis performed using a slab gel method known as pulsed gel electrophoresis. These methods can be useful for any fragment analysis in which the fragment lengths are greater than about 30 kB, which is the size limit for standard agarose gel electrophoresis. RFLP and AFLP fragment size analysis methods are particularly useful for bacterial strain identification and plant hybrid identification, while kerotyping and genetic mapping are generally more useful for research areas of study.

Polynucleic acid analysis kits are also provided by the present invention. These kits include surface neutralized particles with one linker molecule per particle and reagent to link DNA fragment samples to the particles. Although the particles are not necessarily size-fractionated according to the present invention described herein above, without being bound by any particular theory, size-fractionated particles as described herein above are particularly useful because of the higher separation efficiency capability. Suitable STR analysis kits are also provide by the present invention. These kits include forward STR primers attached to neutralized and size-fractionated particles and further include one or more reverse primers, TAQ polymerase, and Mg containing buffer and other reagents useful for conducting PCR.

### EXAMPLES AND OTHER ILLUSTRATIVE EMBODIMENTS

In one example, suitable latex nanospheres used as drag-labels to perform ELFSE for STR analysis include, but are not limited to, the following characteristics: 1) are size fractionated sufficiently to obtain the size homogeneity required for forensic STR analysis; 2) include DNA STR forward primers attached to latex nanospheres; 3) have neutralized surface charge on the latex nanospheres; 4) are capable of performing PCR using forward primers attached to latex spheres with conventional or end-labeled reverse primers; and 5) capable of providing 0.5 base pair (“bp”) resolution at 200 bp; and 6) 0.5 bp resolution at 500 bp.

A protocol for ELFSE analysis is developed. Software for automated analysis of ELFSE STR is also developed to analyze ELFSE data to obtain allele identification for each locus.

Fluorescence “Bar-coding” of Mini-STR Loci is also developed. Fluorescence color and intensity coding system in the latex spheres enables unique “codes” of individual loci when multiple STR loci ranges overlap with fragment sizes up to about 200 bp. A multiplexed system is designed allow a single PCR amplification for all loci.

In a second example, for fragment size analysis, sufficiently size-fractionated latex is attached to a linker molecule and neutralized. This can be incorporated into reagent kits including EDAC and pH buffer that allows attachment to sample DNA fragments for size analysis using ELFSE.

Research Design and Methods—The Size Fractionation of CML Nanospheres. The size homogeneity of CML nanospheres obtained from any of several suppliers (IFD, Molecular Probes/Invitrogen, Bangs Laboratories, Polysciences, and Duke Scientific) ranges from a few percent to about 15 percent. TEM of a collection of such sub-microscopic particles reveals that they are exceedingly uniform in size and shape being near perfect spheres. Although they have uniform size, their size variance still exceeds about 2%. Generally the larger sizes (>1 micron) have lower size variance, but the lot-to-lot variation is such that the smallest size (20 nm) can have a size variance of a few percent in some production lots. When used as an end-label for ELFSE separation of DNA fragments, the limiting resolution due to size inhomogeneity of the nanospheres is the full-width at half-maximum (FWHM) of the size distribution over the average size. The FWHM for a Gaussian is 2.3 times the standard deviation, so a 0.5 bp resolution at 500 bp (0.1% resolution) is achieved using end-labels having a size variance of about 0.04%. For mini-STR about 0.2% resolution is sufficient for 0.5 bp resolution (0.5 bp/250 bp). Size fractionation of commercially obtained CML nanospheres is useful for obtaining size variances less than 2%, preferably less than 1%, or less than 0.5%, or less than 0.2%, less than 0.1%, or even less than 0.05%.

### Example

Reduction in the size variance of 43 nm diameter carboxylate-modified latex (CML) nanospheres from 8% to less than 0.09% has been demonstrated. A 0.7% agarose gel is prepared using the standard method using 0.5× Tris-acetate buffer. After the gel solution is prepared and cooked, it is allowed to cool to 60° C., then 300 micro-liters of 4.5% poly(dimethylacrylamide) is added to 15 mL of agarose gel solution. This is poured before cooling into a 15 cm long, 5 mm I.D. diameter glass tube (7 mm O.D.) with the bottom sitting on a rubber pad to prevent hot gel draining. The top of the tube is slightly modified using a small layer of silicon rubber around the inner surface of the tube to prevent the gel from sliding out of the tube after it has cooled and the bottom is lifted from the rubber pad. After the gel has cooled and set, a 1″ length of ¼″ I.D. PVC tubing is fitting around the top of the tube and a 15 ml centrifuge tube that has the end removed. The centrifuge tube and PVC tubing are filled with approximately 7 mL of cathodic buffer (0.5× Tris-Acetate). For the initial part of the run, the bottom of the gel-containing tube is placed in approximately 10 mL of anodic buffer (0.5× Tris-Acetate). Electrodes are placed several millimeters from each end of the gel and connected to a 250 V power supply.

The CML samples used for fractionation consist of a CML suspension in loading dye. One micro-liter of a 1% CML stock suspension in water is added to 100 micro-liters of 1× loading dye in 0.5× Tris-Acetate buffer for a final concentration of 0.01% CML. Higher concentrations may be advantageous for increased production rate.

After a predetermined run time, fractional collection of the CML exiting the bottom of the tube is obtained. First, the electrophoresis is paused, and the 10 mL of anodic buffer is replaced with a piece of parafilm containing a 30 micro-liter bead of buffer. The end of the gel-containing tube and the electrode are placed in the bead, then the electrophoresis is continued. After a collection time of five minutes or less, the bead is extracted using a pipettor and placed into a 200 micro-liter PCR tube. Then a new bead of 30 micro-liters of buffer is placed on the parafilm and the electrophoresis is resumed. This procedure is repeated for each fraction collected. The first fraction containing measurable CML appears after 75 minutes of run time. Fractions collected after as much as 185 minutes of electrophoresis run time still have measurable amounts of CML.

To verify the reduced size dispersion of the fractionated sample, the collected fractions are run on a capillary electrophoresis system. Homogeneity dispersion may be the primary zone broadening effect for the capillary electrophoresis run. Homogeneity dispersion can be used to determine reduction in size dispersion. When running particles with size variance reduced by the fractionation procedure, other zone broadening effects in capillary electrophoresis can dominate the zone broadening. Even when using a coated capillary, wall interactions dominate zone broadening of the fractionated particles. Because of this diagnostic limitation, the size variance of the particles should be reduced to some minimum acceptable value. Using a poly(acrylamine) coated capillary with 43 nm diameter CML with a size variance of 15% in 1×TBE buffer, the CE system are used to verify if the fractionation procedure reduced the size variance at least a factor of 35 compared to the unfractionated CML. CE systems with lower reduced wall interaction are able to verify lower size dispersion from the fractionation procedure.

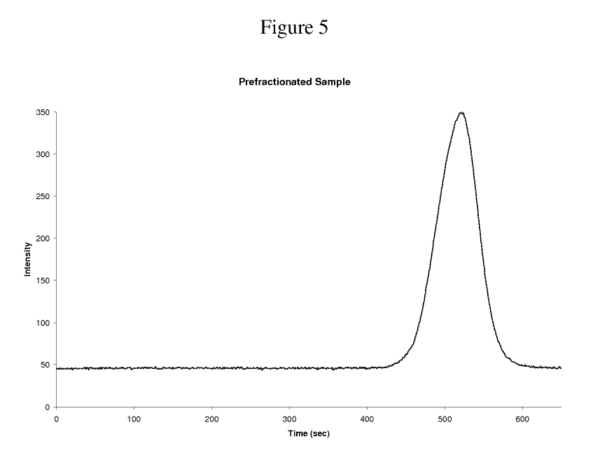

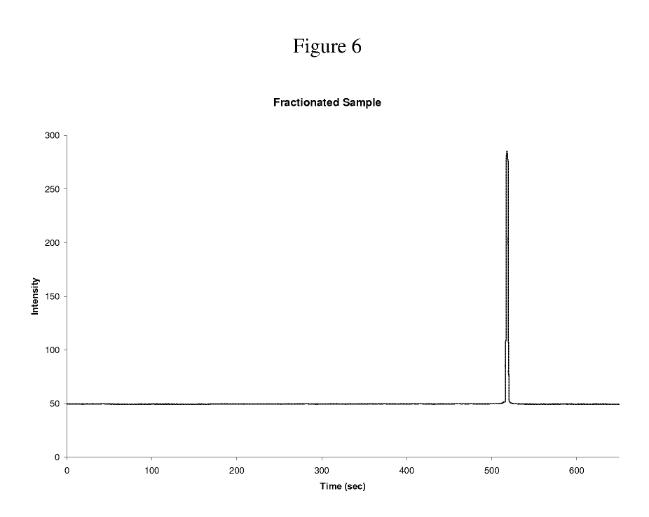

FIG. 5 shows the capillary electrophoresis of the nanospheres before fractionation, and FIG. 6 shows capillary electrophoresis after fractionation using 2 min collection times. The electrophoresis bands of the size-fractionated nanospheres are considerably narrower than the unfractionated nanospheres.

Attachment of STR primers to latex nanospheres. Polynucleic acid oligomers are attached to carboxylate-modified latex using 5′ amino-modified oligomers of various lengths, which are commercially available from a variety of oligomer synthesis companies. Carbodiimide (e.g., EDC) can be used to make a reactive intermediate with the latex surface carboxylate groups, which reacts readily with primary amines, such as the 5′ terminal amine on the oligomer. Commercially available oligomers may also optionally have linking chains, typically C₅ or C₁₀ in length, inserted between the oligomer and terminal amine. This places the oligomer further from the surface after conjugation, which can prevent steric interference with polymerase when used as a primer.

This approach can be used to make end-labeled STR (and mini-STR) primers. A fivefold molar excess of CML nanosphere particles to primer can provide predominately one or no primer attached to each nanosphere. Without being bound by any particular theory of operation, this is consistent with Poisson statistics, which predicts that 80% of the particles will not be conjugated to an oligomer, 18% will have one oligomer attached, and 2% will have two or more oligomers attached. Other methods to ensure that only one oligomer be attached to each CML nanosphere may be devised that make more efficient use of the latex.

Neutralization. After the particles are size fractionated and the primer linked, the remaining carboxylate surface groups are neutralized. Without being bound by any particular theory of operation, it is believed that the carboxylate groups help prevent aggregation of the particles in aqueous suspensions. If they are neutralized, a hydrophilic layer on the surface of the particles can be used to minimize aggregation. Reaction of the surface carboxylate groups with amino-propanol is sufficient for this purpose. The reaction can form a layer of amide propanol on the surface at elevated temperature, (e.g., for about 30 min at 45° C.). After the reaction, injection into a capillary electrophoresis system showed the intensity of the electrophoresis band (see FIG. 5) decreased to about 3% of its original value. Similar results were observed with the reaction rate increasing with temperature observed between 60° C. and 30° C. When the neutralized CML is loaded into an agarose slab gel system, no migrating band of charged nanospheres is observed and the neutralized nanospheres are observed to remain in the loading well after a 3 hr. run. No aggregation of the neutralized nanospheres was observed, even after being stored at 4° C. for several weeks. For use as a drag-label for ELFSE the surface charge is preferably completely removed.

The use of diimide can speed the yield of complete neutralization to less than 30 minutes. The diimide reaction with carboxylates can form an unstable intermediate that reacts readily with primary amines. In the case of amide formation reactions that are slow, the intermediate is stabilized using hydrazine. The reaction of surface carboxylates with amino-propanol may be different. Without being bound by any particular theory of operation, it is believed that the formation of amides from direct reaction of carboxylate with primary amines is probably quite slow and the use of a diimide or conversion of the carboxylate to carbonyl chloride is probably needed to form amides. The use of diimide is the same method typically used to attach amine labeled primer to carboxylate modified latex surfaces. A 97% neutralization by the formation of amide via direct reaction of amino-propanol with CML has been experimentally observed. The use of diimide allows complete reaction of the carboxylate-modified surface to form amide-modified surface to neutralize the nanospheres.

Test of ELFSE using STR primers. Polynucleic-acid-linked particles to be characterized for size using ELFSE are prepared using mini-STR primers that have the 5′ end modified with a C₅ or C₁₀ linker with a terminal primary amine. The end-labels can be size-fractionated CML or any other surface-functionalized particle. End-labeling is carried out by first size fractionating, followed by conjugation of the amino-modified primer and neutralization. Alternatively, conjugation can be performed first, followed by size-fractionation. Both methods can be used. If conjugation affects size variance of the particles it may be preferred to first conjugate the polynucleic acids and particles, and then size fractionating the conjugated product. Either the forward or reverse primer, or both, can be conjugated to a CML particle before being used for PCR.

The neutralization step with amino-propanol is not expected to affect the size variance of the nanospheres. The carboxylate group surface density is high (about 66 carboxylate groups per square nm for CML have been tested). Accordingly, 1-hydroxyl-propylamide surface groups apparently can be oriented in a radial manner with respect to the spherical surface. Variance in surface group/linker length at the surface is typically negligible.

Study PCR on primer attached to latex spheres. The polynucleic acid primer is attached to a “tether” to move it far enough away from the surface to avoid steric hindrance of the polymerase. Although kinetics may be slower as the smaller, faster moving primer is now attached and the heavier, slower moving template is moving freely, once hybridized, though, the polymerization rate should be similar to the situation where the DNA template is bound to the surface. In addition, PCR amplification can occur in solution where template and particle-linked primer are freely moving.

Once the mini-STR primers are linked to suitable particles, and the resulting particles have a size variance less than 2%, ELFSE resolution is tested using an STR allelic ladder, in a manner similar to the testing of mini-STRs. In this method, commercially available STR allelic ladder is diluted and used as template for mini-STR primer. The mini-STR primers anneal to appropriate STR alleles.

In conducting ELFSE mini-STR resolution, a single mini-STR primer is used to form allelic ladder for the corresponding mini-STR locus. The resulting product is analyzed using a single-capillary electrophoresis system in a denaturing buffer to determine the resolution. Using a mini-STR locus with a fragment length of about 150 bp, a relative resolution of 1:300 or 0.3% demonstrates 0.5 bp resolution. Further optimization of the electrophoresis conditions, including injection time and method used (hydrodynamic vs. electrokinetic), run voltage, capillary length, coated vs. uncoated capillary, and the like, is readily carried out.

Two mini-STR primers corresponding to two loci with overlapping range of alleles with the same nanospheres (color and size) are also be prepared and used. After PCR amplification of allelic ladder, the product is run on a single capillary electrophoresis system. This determines if different loci having a relative mobility shift. No mobility shift occurs if the same end-label particles are used for each locus.

Particles of two different colors of the same size, are used as drag labels for polynucleic acids. To accomplish this, the same primer is conjugated to two sets of particles of different colors but same nominal size, then combined and size fractionated together. A single fraction, containing both colors, can then be neutralized, and PCR amplified using STR allelic ladder as template. No mobility shift is observed for the STR ladder with different color labels.

Different fractions from a particular particle size fractionation are combined to give rise to a mobility shift of the allelic ladder.

Mini-STR loci are linked to nanospheres and size-fractionated to give rise to 0.5 bp resolution for STR analysis using ELFSE.

Example Demonstrating 0.5 bp Resolution for Standard STR Analysis—Reducing size-variance using tube gel fractionation. For standard STR analysis, the fragment lengths of alleles can approach 500 bp. To obtain 0.5 bp resolution, the relative resolution required is 1:1000. To achieve the required size homogeneity of the nanospheres, the tube gel electrophoresis system and operating parameters are further modified. Additional modifications include a longer free solution electrophoresis tube, water cooling of the tube, and increased agarose concentration. It is desirable to minimize tube length and agarose concentration to minimize the required separation time.

Once the required size fractionation is obtained, a set of tests using allelic ladder similar to the tests performed for the mini-STR 0.5 bp resolution is carried out.

Using a commercially-available mini-STR multiplex system, three sets of latex spheres, each with a different fluorescence color, are used to label three sub-sets of primers STR. These latex spheres can also be used to label primer for allelic ladder. Latex spheres having a fourth fluorescent color is labeled with primer and used as an internal size standard.

Size-fractionating all colors (multiplexes) simultaneously. Once primer is attached to the latex spheres at a surface carboxylate group, the spheres are size fractionated to obtain a desired separation resolution. Commercially obtained latex spheres having a size variance in the range of a few percent up to 10% can be used. One example is carboxylate-modified spheres having an average size of 42 nm obtained from Interfacial Dynamics Corp., were measured by capillary electrophoresis having a size variance of 3.5%. The size variance of these nanospheres is reduced by using a tube gel electrophoresis system with 0.7% agarose in which size fractions are collected in small volumes of anodic buffer.

Mini-STR Concordance. Concordance of ELFSE mini-STR analysis with standard CGE STR analysis includes two studies. The first compares results using a single locus mini-STR system. Then multiplexed samples using particle-linked primers are used. The multiplexing identifies the allelic ladder and the allele of the locus made from the same set of size fractionated particles, which avoids relative mobility shifts.

Reagent Preparation. An allelic ladder is prepared as described previously using commercially available STR allelic ladder as template. Size standards are prepared from commercially available DNA ladder and end-labeled by first aminating the 5′ end of the ladder fragments, followed by end-labeling and neutralization as previously described. Particles from the same size fractionation are used with allelic ladder and unknown/sample primer of one locus to avoid mobility shift.

Color-Coded Spheres. Another advantage of latex spheres is that different intensities of different dyes are impregnated into the polystyrene nanospheres, which adds another dimension to multiplexing. For example a three-color system with three intensity levels (0, ½, and 1) allows 26 unique “codes” to be produced in different spheres. Different codes are used to identify each STR locus and the primer for that locus is attached to nanospheres with its unique color-code, allowing identification of alleles from 25 different loci and an internal lane standard.

Color-coded (“Bar-coded”) particles are used to allow multiplexing of all loci using particles with sixteen different “bar-codes” into a single PCR reaction. Each code can include a unique pattern of one or more of four colors. The four colors are desirably selected to be compatible with one of the color filter sets on a suitable commercially-available STR capillary system, e.g., Advanced Biosystems.

Concordance of ELFSE STR Results with CGE Results. ELFSE standard STR analysis using commercial primers is conducted using size fractionated particles having a sufficiently reduced size variance capable of demonstrating 0.5 bp resolution. This is less complex that the mini-STR concordance studies, because the multiplexing is similar to CGE multiplexing. Just as for the mini-STR, the same size fractions are used for the allelic ladder and corresponding primer. For the mini-STR system this is straightforward, because the same primer can be used to make the allelic ladder and amplify the DNA locus. A commercial allelic ladder can be used as a template to make end-labeled allelic ladder with the end-labeled primer. Commercially-available primers for PowerPlex 16 can be end-linked to a particle using an aminated primer as for the mini-STR system. One difference is that the primers can anneal to the ends of the allelic ladder.

Analysis Software. Genetics analysis software is provided to convert to analyze ELFSE migration times to fragment size and perform size analysis using DNA internal lane size standards and allelic ladder for each STR locus. This software allows for automated analysis, making allele calls for all the loci.

Degraded Samples. Partial or no STR profiles from degraded samples recovered at the site of the World Trade Center gave rise to the development of mini-STR analysis. This STR technique uses primers designed to lie down on the genomic DNA closer to the repeat elements of STR loci, resulting in shorter allelic fragments. This technique which can use STR primers incorporated into multiplexes provides increased ability to obtain profiles from degraded DNA using skeletal samples

In addition to improved analysis of degraded samples using thirteen core loci, six non-CODIS STR loci that result in fragments less than 125 bp have been characterized. Profiles of degraded genomic DNA samples are obtained using the 41 additional non-CODIS loci that have been identified. Adapting shorter fragment lengths using the mini-STR technology relies on the range of possible allele fragment sizes for each of the loci in a color set to not overlap. Although the original STR loci for the CODIS loci are contained in three or four colors with four to six loci per color, further reduction of the fragment size, which would result in an even more powerful ability to analyze degraded DNA samples, is limited because of allelic overlap. To address this problem, more colors are used, but this is limited by instrumentation capabilities. The fluorescent “bar-code” system greatly improves the ability to institute the mini-STR system as previously described, keeping the alleles of all loci less than 125 bp without having to keep the size range of alleles from different loci from overlapping.

Reduced Need for Quantification. Another area of potential improvement in STR analysis is elimination of the requirement to quantify the DNA to ensure that a viable sample is present and that the quantity of amplified DNA falls within the dynamic range of the fluorescence detection system. Less than 0.5 ng of DNA may not produce a full profile, and more than 2 ng may saturate commercial detection systems e.g., an AB 310, 3100 or 3130xl by Applied Biosystems using standard PCR protocol, or result in stochastic effects. Systems with increased sensitivity allow for the identification of samples with reduced quantities of template DNA or fewer PCR cycles, which could decrease the total process time. Without being bound by any theory of operation, because large amounts of fluorescent dye can be impregnated into the latex spheres (ca. 250 dye molecules in a 40 nm sphere), sensitivity is greatly enhanced. It is relatively easy to detect single dyed nanospheres in a capillary using an optimized detector, so detection of single end-labeled DNA molecules is possible according to the ELFSE methods of the present invention. In addition, by using end-labeled primer as the limiting reagent in PCR, saturation of the detection system is avoided, which may reduce or even eliminate the need for quantification.

Reducing Use of Sieving Matrix. One of the costs of running an STR analysis is the sieving matrix. A method of STR analysis that does not require sieving matrix could reduce the cost of STR analysis. Shortening the time to perform the electrophoresis step will reduce a bottleneck, and may even allow a laboratory to use fewer instruments or test greater numbers of DNA samples.

Compatibility with Existing Instrumentation. The ELFSE STR analysis system, which includes fluorescent dye-impregnated latex spheres with primer attached, can be compatible with all capillary electrophoresis instruments used by the forensic community. The sieving matrix used in these instruments is not used for ELFSE, resulting in a cost savings. Also, the run times can be a factor of two or three shorter than when a sieving matrix is used.

Polynucleic acid size analysis other than STR. Polynucleic acid fragments resulting from fragmentation analysis techniques such as RFLP, AFLP, karyotyping, and the like, can be size analyzed using the ELFSE technique with CML as the drag-labels. In this method, the polynucleic acid fragments are first modified at the 5′ end by adding a primary amine terminal unit, using standard techniques. The CML, in some cases size-fractionated and in other cases used with the size dispersion from the emulsion polymerization process (about 2% to 10%), is conjugated to the 5′ amino-modified polynucleic acid fragments with the aid of diiminide as described previously, then neutralized as described previously. Using this method, fragment sizes can be separated using ELFSE that cannot be separated by standard gel electrophoresis (about 30 kB), which typically is preformed using pulsed gel electrophoresis with run times of tens of hours. The use of highly fluorescent CML will allow very small amounts of polynucleic acid to be fragment-size separated with single molecule detection as a possibility.

Potential for Miniaturization. The ELFSE STR reagent system is completely compatible with “lab on a chip” technology, since free solution electrophoresis gives the same results when run on a miniature system, e.g., wherein DNA isolation and PCR are integrated on the same “on-chip” platform. This lends itself well for portable applications to realize a presumptive DNA screening approach and possibly to real-time crime scene DNA analysis.

Discussion of Results. Methods to reduce the size variance of particles for use in genomic analysis is provided. In one embodiment carboxylate modified latex to amide modified latex with a hydrophobic end group is neutralized and converted reduced size variance particles, e.g., nanospheres, with neutralized surface for genomic analysis (excluding sequencing) are provided. Standard emulsion polymerization to form latex gives rise to, a size variance of 2% to 15% is obtained under good conditions. The main factor contributing to zone broadening under optimal capillary electrophoresis conditions to reduce wall effects, diffusion, and injection width is size variance of the sample.

Without being bound by any particular theory of operation, the drag is proportional to the square of the particle diameter (not linear as with standard CE) in gel electrophoresis. If the surface charge did not have double layer, the drag to charge ratio would not change with size. Because the double layer causes the effective surface charge to decrease with size (more effective double layering), then the larger fragments migrate more slowly. The advantage of using gel electrophoresis is that a tube several millimeters in diameter can be used, which allows much larger sample volumes to be used.

For fragment analysis of large fragments, larger particle sizes can be used; up to about 10 microns can be used with a 50 micron ID capillary, which can allow separation of fragments up to about 300 kBases; up to about several mega-bases (“MBases”) would use particles about 100 microns in diameter.

Tube gel electrophoresis systems provided herein obtained a particle enables ELFSE size variance less than 0.1% (defined as standard deviation of size distribution divided by the average size). STR applications using particle “drag labels” having a 0.5 base resolution for fragment sizes up to 500 bases can be achieved using the present invention.

What is claimed:

1. A method for preparing neutralized polynucleic acid-attached particles, comprising:
size fractionating a plurality of charged surface-functionalized particles to provide a plurality of size-fractionated charged surface-functionalized particles being characterized as having a size variance of less than 2 percent;
neutralizing at least a portion of the size-fractionated charged surface-functionalized particles with a hydrophilic neutralizing agent to give rise to neutralized size fractionated particles being characterized as having a hydrophilic surface; and
attaching a polynucleic acid, directly or indirectly, to each of at least a portion of the neutralized size fractionated particles, or to each of at least a portion of the size-fractionated charged surface-functionalized particles, to provide neutralized polynucleic acid-attached particles.

2. The method of claim 1, wherein each polynucleic acid is attached indirectly to at least a portion of the neutralized size-fractionated particles via a linker molecule attached to the size-fractionated charged surface-functionalized particles before neutralization.

3. The method of claim 1, wherein each polynucleic acid is attached directly to at least a portion of the size-fractionated surface-functionalized particles via covalent bonding before neutralization.

4. The method of claim 1, wherein the charged surface-functionalized particles are labeled or impregnated with one or more fluorescent dye molecules, energy-transfer donor-acceptor fluorescent dye molecule pairs, quantum dots, or any combination thereof.

5. The method of claim 4, wherein the fluorescent dye molecules, energy transfer dye molecules, or quantum dots, or any combination thereof, emit at two or more different wavelengths with different intensities resulting in fluorescent pattern signatures.

6. The method of claim 1, wherein the charged surface-functionalized particles are size fractionated using electrophoresis, chromatography, field-flow fractionation, or other general types of charge, size, or charge/size fractionation processes, or any combination thereof.

7. The method of claim 6, wherein the electrophoresis uses an agarose gel, nanofabricated physical sieving device, or other sieving matrix, or any combination thereof.

8. The method of claim 1, wherein the charged surface-functionalized particles comprise charged surface-functionalized latex particles, wherein the latex particles comprise a polymeric material composed of repeating units derived from styrenic monomers, acrylic monomers, acrylonitrile monomers, diene monomers, or any combination thereof.

9. The method of claim 1, wherein the charged surface-functionalized particles comprise one or more functional groups grafted to the surface of the charged surface functionalized particles carboxylic acids, the functional groups comprising aliphatic amines, aromatic amines, hydrazide, sulfate, acidic functional groups, basic functional groups, or any combination thereof.

10. The method of claim 1, wherein the plurality of charged surface-functionalized particles prior to fractionating are characterized as having an average particle size in the range of from about 5 nm to about 500,000 nm and a size variance distribution of greater than about 2%.

11. The method of claim 1, wherein the plurality of size-fractionated, charged surface-functionalized particles are characterized as having an average particle size of greater than about 5 nm and a size variance of less than about 2 percent.

12. The method of claim 2, wherein the linker molecules include difunctionalized primary amines for bonding to carboxy or other acidic surface modification, or primary amines comprising carboxylic acid for amine surface-modification, or any combination thereof,
wherein the difunctionalized primary amines include any compound having the structure
X′—Y—X″
wherein X′ and X″ are functional groups and Y is an alkyl group comprising from 1 to about 40 carbon atoms and from 2 to about 80 hydrogen atoms.

13. The method of claim 12, wherein X′ is a primary amine for attachment to the polynucleic acid and X″ is a carboxylate, amine, sulfate, or thiol group for attachment to the charged surface functional groups of the neutralized size-fractionated surface-functionalized particles.

14. The method of claim 1, wherein the size-fractionated, charged surface-functionalized particles are neutralized by converting the charged surface functional groups to an uncharged hydrophilic group by reaction with a hydroxyl-bearing compound, amide-bearing compound, aldehyde-bearing compound, thiol-bearing compound, or any combination thereof.

15. The method of claim 14, wherein the hydrophilic group includes a hydroxyl group, amide group, aldehyde group, thiol group, or any combination thereof.

16. The method of claim 15, wherein the charged size-fractionated surface-functionalized particles are neutralized by reacting at least a portion of the charged surface functional groups with a hydroxyl-bearing compound, amide-bearing compound, aldehyde-bearing compound, thiol-bearing compound, or any combination thereof, and wherein the hydroxyl-bearing compound comprises 3-aminopropanol.

17. The method of claim 1, wherein at least a portion of the size-fractionated charged surface-functionalized particles are neutralized, wherein particles that are less than fully neutralized are removed using electrophoresis.

18. The method of claim 1, wherein the polynucleic acid comprises a PCR primer, a DNA fragment, a RNA fragment, or any combination thereof.

19. A method, comprising:
providing a plurality of primer-linked particles comprising PCR primer molecules, each labeled with a neutralized particle characterized as having an average particle size in the range of from about 5 nm to about 100 nm, each particle having a size variance of less than about 0.5 percent; and
amplifying a DNA sample using the primer-linked particles in the presence of PCR reagents to give rise to particle-labeled amplicons; and
performing size analysis of the particle-labeled amplicons using capillary electrophoresis.

20. The method of claim 19, wherein the attached primers are one of the primers in a primer pair for amplification of STR loci used in forensic identification.

21. The method of claim 19, wherein fewer than 10 thermocycles are performed to amplify the DNA.

22. The method according to claim 19, wherein the capillary electrophoresis includes ELFSE comprising a device, the device comprising a capillary, a microfluidic channel, or any combination thereof, wherein the particle-labeled amplicons flow through a capillary or microfluidic channel length of between about 1 cm and about 100 cm, in the presence of an applied voltage field in the range of from about 50 V/cm to about 2000 V/cm.

23. A polynucleic acid sample analysis reagent kit, comprising
a plurality of size-fractionated, surface-neutralized particles having an average particle size in the range of from about 5 nm to about 500 microns, the size variance of the particles being less than about 0.5 percent, at least a portion of the particles being attached to a PCR primer or a linker molecule; and
a reagent to link a polynucleic acid to each of the linker molecules.

24. The polynucleic acid analysis kit of claim 23, wherein the polynucleic acid comprises one or more DNA fragments obtained from a biological sample, or a synthetic PCR primer.

25. A method of analyzing DNA fragments by size, comprising:
attaching a plurality of surface neutralized particles to sample DNA fragments, wherein the surface neutralized particles have an average particle size in the range of from about 5 nm to about 500 microns, the size variance of the particles being less than about 0.5 percent, wherein at least a portion of said particles are attached to a PCR primer or linker molecule capable of being attached to a polynucleic acid using reagents to provide DNA-linked particles; and
analyzing the DNA-linked particles using ELFSE.
